(19) United States
(12) Patent Application Publication
(10) Pub. No.: US 2001/0023259 A1
(43) Pub. Date: Sep. 20, 2001

(54) Conjugated fatty acids and related compounds

(75) Inventors: Antoni Ryszard Slabas, High Shincliff (GB); Josiah William Simon, High Shincliff (GB); William Walker Christie, Dundee (GB)

Correspondence Address: J. Mitchell Jones, MEDLEN & CARROLL, LLP, Suite 2200, 220 Montgomery Street, San Francisco, CA, 94104, US

(73) Assignee: Natural ASA

(21) Appl. No.: 09/740,145

(22) Filed: Dec. 18, 2000

(30) Foreign Application Priority Data
Dec. 18, 1999 (GB) 9929897.8

(51) Int. Cl. A61K 31/202; A61K 31/22

(52) U.S. Cl. 514/560; 514/552; 554/230

(57) ABSTRACT

It has been demonstrated that conjugated linoleic acid isomers with the first double bond in position 9 (cis) or 10 (trans), added exogenously, can be desaturated in position 6 by the cyanobacterium Spirulina platensis. The metabolites, 6-cis, 9-cis, 11-trans-octadecatrienoic and 6-cis,10-trans,12-cis-octadecatrienoic acids, which have not previously been characterized, were isolated by a combination of chromatographic techniques and the structures were confirmed by gas chromatography-mass spectrometry in the form of picolinyl ester and dimethyloxazoline derivatives.

This application claims priority under 35. U.S.C. § 119 (a)-(d) to United Kingdom application 99 29 897.8, filed Dec. 18, 1999.

### FIELD OF THE INVENTION

This invention relates to a novel method of making certain compounds (especially fatty acids and derivatives thereof) being desaturated at a 6th carbon atom in a chain of carbon atoms, relative to the starting substrate; certain novel compounds being unsaturated at a 6th carbon atom in a chain of carbon atoms; and to compositions for nutritional and/or pharmaceutical use, comprising certain fatty acid compounds and derivatives thereof. The invention also provides for use of certain compounds as nutritional supplements and/or pharmaceuticals; and a method of making a nutritional and/or pharmaceutical composition.

### BACKGROUND OF THE INVENTION

Octadecadienoic acid is the name given to C18 fatty acids having two carbon/carbon double bonds (i.e., C_{18:2} fatty acids). The carbon/carbon double bonds may be positioned essentially at any point along the hydrocarbon chain (other than, of course, involving the carbon atom of the carboxyl group). Linoleic acid is the name given to the octadecadienoic acid having carbon/carbon double bonds at positions 9 and 12, both being in the “cis” configuration (i.e., cis-9, cis-12 octadecadienoic acid). Those skilled in the art will appreciate that the two carbon/carbon double bonds, when separated by one carbon/carbon single bond, may form a small “conjugated” system of delocalized electrons in the carbon atoms. Such molecules may be referred to as conjugated linoleic acid (abbreviated as CLA), even though the term “linoleic acid”, strictly speaking, refers only to the cis-9, cis-12 compound. It will be apparent that there are many possible isomers of CLA depending, for example, on the position of the double bonds (“positional isomers”), or on the stereochemistry (“geometric isomers”) of the double bonds (which may be trans/trans, cis/cis, cis/trans or trans/cis; abbreviated as t/t, c/c, c/t and t/c respectively).

The term “CLA” has hitherto conventionally been used primarily to refer to 9-cis,11-trans-octadecadienoic acid, which is a minor component of milk, dairy products and ruminant fats (<1%), and has long been recognized as having anti-cancer properties (Pariza & Hargraves Carcinogenesis 6: 591-593 (1985)). Subsequently, it was claimed to have anti-atherosclerosis effects, to help the immune system and to affect energy metabolism, promoting deposition of protein rather than fat. The biological effects of CLA are well documented and have been reviewed comprehensively (Banni & Martin, Trans Fatty Acids in Human Nutrition (ed. J. L. Sébédio and W. W. Christie, Oily Press, Dundee), pp. 261-302 (1998)), and it is now apparent that more than one isomer may be involved (especially 10-trans,12-cis-octadecadienoic acid). However, the mechanism of these effects has not been established. Various suggestions have been put forward, one of which is that CLA and its isomers are elongated and desaturated to form analogues of arachidonic acid, which interfere with eicosenoid metabolism; such analogues have been identified in essential-fatty acid-deficient rats fed high doses of CLA (FIG. 1) (Sébédio et al, Biochim. Biophys. Acta, 1345: 5-10 (1997)). Thus, linoleic acid is desaturated to γ-linolenic acid (the rate-limiting step), and then is converted by chain-elongation and further desaturation to arachidonic acid. Two CLA isomers have been shown to be converted to arachidonate analogues in the same way (Sébédio et al, Biochim. Biophys. Acta, 1345: 5-10 (1997)), although the putative C₁₈ intermediates have not been characterized in animal tissues, presumably because they are produced slowly in the rate-limiting step and then rapidly metabolized.

Both of the most readily available CLA isomers (i.e., 9-cis, 11-trans and 10-trans, 12-cis) are present in substantially equimolar amounts in commercial preparations of CLA, which are generally prepared by heating natural linoleic acid (or oils enriched in this) to high temperatures in the presence of alkali, although variable amounts of positional and geometrical isomers may also be present (Christie et al, Lipids, 32, 1231 (1997).

The cyanobacterium Spirulina platensis (also known as Arthrospira platensis) is grown on a large scale and then sold in freeze-dried form in health food shops, because of its high content of protein, vitamins, minerals and especially of γ-linolenate, the active component of evening primrose oil. The organism has a Δ6 desaturase and is able to desaturate endogenous cis-9, cis-12 linoleic acid to cis-6, cis-9, cis-12 γ-linolenate. More surprisingly, it has been shown to be capable of desaturating linoleate added exogenously (Quoc et al, Plant Physiol. Biochem., 32, 501-509 (1994)). A priori, it would not be expected to desaturate fatty acids other than its natural substrate, as unsaturated fatty acids (in free form) are usually highly toxic to microorganisms. Nor does it desaturate oleate, which is also present in the organism, at position 6. The presence of trans double bonds confers on fatty acids a three dimensional structure analogous to saturated fatty acids (e.g., such as oleic acid). Accordingly, the person skilled in the art would not expect the Δ6 desaturase to act on substrates containing a trans carbon/carbon double bond.

### SUMMARY OF THE INVENTION

This invention relates to a novel method of making certain compounds (especially fatty acids and derivatives thereof) being desaturated at a 6th carbon atom in a chain of carbon atoms, relative to the starting substrate; certain novel compounds being unsaturated at a 6th carbon atom in a chain of carbon atoms; and to compositions for nutritional and/or pharmaceutical use, comprising certain fatty acid compounds and derivatives thereof. The invention also provides for use of certain compounds as nutritional supplements and/or pharmaceuticals; and a method of making a nutritional and/or pharmaceutical composition.

Accordingly, in some embodiments, the present invention provides compositions comprising a conjugated octadecatrienoic acid moiety, wherein the conjugated octadecatrienoic acid moiety is desaturated at position 6. The present invention is not limited to any particular isomer of conjugated linoleic acid. Indeed, it is contemplated that the conjugated linoleic acid moiety can include a variety of isomers of octadecatrienoic acid, including, but not limited to, c-6, c-9, t-11 octadecatrienoic acid, c-6, c-9, c-11 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid. Likewise, the present invention is not limited to any particular octadecatrienoic acid moiety. Indeed, a variety of moieties are contemplated, including, but not limited to free fatty acids, esters such as alkyl esters (e.g., methyl and ethyl esters) and triglycerides. In some preferred embodiments, the octadecatrienoic acid moiety is covalently attached to a glycerol molecule.

It is contemplated that the conjugated octadecatrienoic acid moieties of the present invention may be formulated in a variety of ways. In some embodiments, the compositions are formulated with an antioxidant. In other embodiments, the conjugated octadecatrienoic acid is combined with a food product.

In still further embodiments, the present invention provides pharmaceutical compositions comprising an excipient and a conjugated octadecatrienoic acid moiety, wherein the conjugated octadecatrienoic acid moiety is desaturated at position 6. The pharmaceutical compositions of the present invention are not limited to any particular isomer of conjugated linoleic acid. Indeed, it is contemplated that the conjugated linoleic acid moiety can include a variety of isomers of octadecatrienoic acid, including, but not limited to, c-6, c-9, t-11 octadecatrienoic acid, c-6, c-9, c-11 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid. Likewise, the present invention is not limited to any particular octadecatrienoic acid moiety. Indeed, a variety of moieties are contemplated, including, but not limited to free fatty acids, esters such as alkyl esters (e.g., methyl and ethyl esters) and triglycerides. In some preferred embodiments, the octadecatrienoic acid moiety is covalently attached to a glycerol molecule.

In some embodiments of the present invention, compositions comprising a conjugated octadecatrienoic acid moiety are provided that are made by the process comprising: a) providing a conjugated octadecadienoic acid and a delta-6 desaturase; and b) exposing the conjugated octadecadienoic acid to the delta-6 desaturase under conditions such that a conjugated octadecatrienoic moiety desaturated at position 6 is produced.

In other embodiments, the present invention provides methods for preparing a conjugated octadecatrienoic acid moiety comprising: a) providing a conjugated octadecadienoic acid and a delta-6 desaturase; and b) exposing the conjugated octadecadienoic acid to the delta-6 desaturase under conditions such that a conjugated octadecatrienoic moiety desaturated at position 6 is produced. In preferred embodiments, the exposing step further comprises incubating the octadecadienoic acid with a culture of Spirulina platensis. The present invention is not limited to the desaturation of any particular octadecadienoic acid isomer. Indeed, the desaturation of a variety of octadecadienoic acid isomers is contemplated, including, but not limited to c9,t11-, t9,c11-, t9,t11-, c9,c11-, t10,c12-, c10,t12-c10,c12-, and t10,t12-octadecadienoic acids. The present invention is not limited to delta-6 desaturases from any particular source. In some preferred embodiments, the delta-6 desaturase is derived from The present invention is not limited to the use of any particular strain of Spirulina platensis. Indeed, the use of a variety of strains is contemplated, including, but not limited to those designated PCC8005, PCC6313, PCC7345, and PCC7939. In still further embodiments, the methods of the present invention comprise step c) purifying the conjugated octadecatrienoic acid moiety.

In other embodiments, the present invention provides a composition comprising a delta-6 desaturase, octadecadienoic acid, and an octadecatrienoic moiety. In still further embodiments, the compositions additionally comprise a culture of Spirlina platensis.

In some embodiments, the present invention provides methods of altering arachidonic acid metabolism in a subject comprising: a) providing a subject and a conjugated octadecatrienoic acid moiety, wherein said conjugated octadecatrienoic acid moiety is desaturated at position 6; and b) administering the conjugated octadecatrienoic acid moiety to the subject under conditions such that an arachidonic acid analog is formed. The present invention is not limited to use of moieties containing any particular isomer of conjugated octadecatrienoic acid. Indeed, the use of a variety of isomers is contemplated, including, but not limited to c-6, c-9, t-11 octadecatrienoic acid, c-6, c-9, c-11 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid. The present invention is not limited to the formation of any particular arachidonic acid analog. Indeed, the formation of a variety of arachidonic acid analogs is contemplated, including, but not limited to c-5, c-8, c-11, t-13 eicosatetraenoic acid and c-5, c-8, t-12, c-14 eicosatetraenoic acid. The present invention is not limited to any particular dose of the conjugated octadecatrienoic acid moiety. In preferred embodiments, the conjugated octadecatrienoic moiety is provided at a dose of about 10 mg to 10 g per day. The present invention is not limited to any particular octadecatrienoic acid moiety. Indeed, a variety of moieties are contemplated, including, but not limited to free fatty acids, esters such as alkyl esters (e.g., methyl and ethyl esters) and triglycerides, and salts. In some preferred embodiments, the octadecatrienoic acid moiety is covalently attached to a glycerol molecule.

In still other embodiments, the present invention provides novel compounds of the structure:

R₁—O—R₂

wherein R₁ is selected from the group consisting of c-6, c-9, t-11 octadecatrienoyl, c-6, c-9, c-11 octadecatrienoyl, c-6, t-9, t-11 octadecatrienoyl, c-6, t-9, c-11 octadecatrienoyl, c-6, t-10, c-12, octadecatrienoyl, c-6, c-10, t-12, octadecatrienoyl, c-6, t-10, t-12, octadecatrienoyl, and c-6, c-10, c-12, octadecatrienoyl and

R₂ is selected from the group consisting of hydrogen, alkyl, glycerol, and phosphoglycerol. In some embodiments, the alkyl is selected from the group consisting of methyl, ethyl and propyl.

In further embodiments, the present invention provides methods for producing conjugated octadecatrienoic acid comprising a) providing gamma-linoleic acid and a catalyst; and b) reacting said gamma-linolenic acid with said catalyst under conditions such that c6, t10, c12 octadecatrienoic acid is produced. The present invention is not limited to any particular source of gamma-linolenic acid. Indeed, a variety of sources of gamma-linolenic acid are contemplated, including, but not limited to borage oil and evening primrose oil.

In some embodiments, the present invention provides methods of producing a molecule which, relative to a substrate, is desaturated at the 6^{th} carbon atom in a chain of carbon atoms, the method comprising steps of: contacting the substrate with a (6 desaturase enzyme obtainable from Spirulina spp. in suitable conditions so as to cause formation of a product being desaturated at the 6^{th} carbon atom; and collecting the product. In some embodiments, the substrate is an unsaturated fatty acid. In some preferred embodiments, the substrate comprises a trans carbon/carbon double bond. In some other preferred embodiments, the substrate is a di-unsaturated fatty acid and the product is the corresponding tri-unsaturated fatty acid. In preferred embodiments, the substrate is a C14-C20 compound. In some particularly preferred embodiments, the substrate is a C18 fatty acid. In some embodiments, the performance of the preceding methods results in the production of one or more of the following: 6-c, 9-c, 11-t octadecatrienoic acid; 6-c, 10-t, 12-c octadecatrienoic acid; and octadeca-6,9-dien-12-ynoic acid. In other embodiments, the methods comprise contacting the substrate with a plurality of cells of Spirulina organisms. In still further embodiments, the product is in the form of a fatty acid, lipid or other ester.

In some embodiments, the present invention provides the following compounds: the compound 6-c, 9-c, 11-t octadecatrienoic acid or a derivative thereof; the compound 6-c, 10-t, 12-c octadecatrienoic acid or a derivative thereof, and the compound octadeca-6,9-dien-12-ynoic acid or a derivative thereof. In some embodiments, these compounds are derivatized at the carboxyl group. The present invention is not limited to any particular derivative. Indeed, a variety of derivatives are contemplated, including, but not limited to a salt, ester, amide or aldehyde.

In some preferred embodiments, the present invention provides compositions suitable for administration to a mammalian subject, comprising at least 0.001% w/w, 0.01% w/w, 0.1% w/w, or 1.0% w/w of the compounds described previously.

In other preferred embodiments, the present invention provides a pharmaceutical or nutritional composition suitable for administration to a mammalian subject comprising a Δ6 unsaturated fatty acid or physiologically acceptable derivative thereof, together with a physiologically acceptable excipient, bulking agent or diluent. In some preferred embodiments, the compositions are in the form of a tablet or capsule for oral administration to a subject.

In still other embodiments, the present invention provides methods of making a composition for consumption by a mammalian subject, the method comprising the steps of providing a Δ6 unsaturated fatty acid or physiologically acceptable derivative thereof, mixing the Δ6 unsaturated fatty acid or derivative thereof with a suitable excipient, bulking agent or diluent; and packaging the resulting mixture. In further embodiments, the present invention provides for the use of a Δ6 unsaturated fatty acid or physiologically acceptable derivative thereof in the manufacture of a medicament or nutritional supplement to be administered to a mammalian subject. In still further embodiments, the present invention provides for the use of a Δ6 unsaturated fatty acid or physiologically acceptable derivative thereof in the manufacture of a medicament to inhibit one or more actions of arachidonic acid in a mammalian subject.

In some embodiments, the present invention provides methods of producing a molecule which, relative to a substrate, is desaturated at the 6^{th} carbon atom in a chain of carbon atoms, substantially as hereinbefore described. In other embodiments, the present invention provides a Δ6 tri-unsaturated fatty acid substantially as hereinbefore described and with reference to the accompanying drawings. In still other embodiments, the present invention provides pharmaceutical or nutritional compositions suitable for administration to a mammalian subject substantially as hereinbefore described.

### DESCRIPTION OF THE DRAWINGS

FIG. 1 shows the pathway by which linoleic acid may be converted into arachidonic acid in mammalian tissues, and the pathways by which two particular CLA isomers may be converted into analogues of arachidonic acid;

FIG. 2A shows the structural formula of a picolinyl ester derivative of 6-c, 10-t, 12-c octadecatrienoic acid, and FIG. 2B shows the mass spectrum obtained for this compound;

FIG. 3A shows the structural formula of a 4,4 dimethyloxazoline derivative of 6-c, 10-t, 12-c octadecatrienoic acid, and FIG. 3B shows the mass spectrum obtained for this compound;

FIG. 4A shows the structural formula of a picolinyl ester derivative of octadeca-6,9-dien-12ynoic acid, and FIG. 4B shows the mass spectrum obtained for this compound.

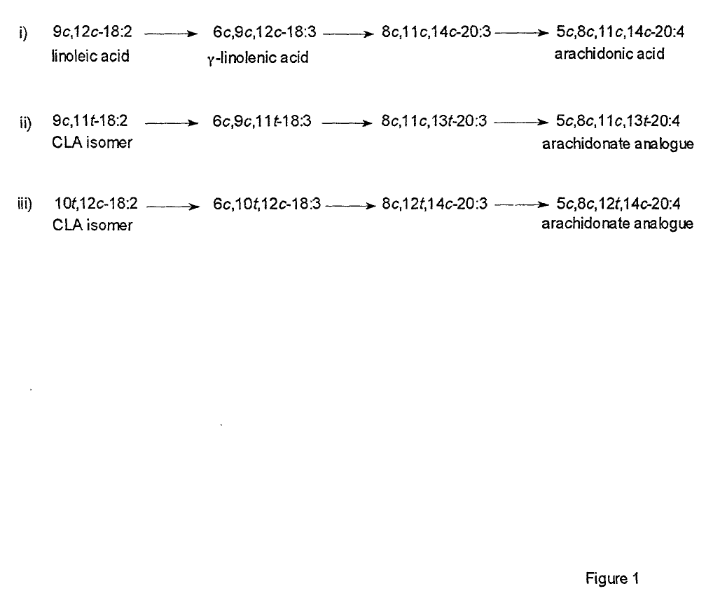

### DEFINITIONS

As used herein, “conjugated linoleic acid” or “CLA” refers to any conjugated linoleic acid or octadecadienoic free fatty acid. Likewise, the term “conjugated octadecatrienoic acid” refers to any octadecatrienoic free fatty acid that contains conjugated double bonds. It is intended that these terms encompass and indicate all positional and geometric isomers of linoleic acid and octadecatrienoic acid with two conjugated carbon-carbon double bonds any place in the molecule. CLA differs from ordinary linoleic acid in that ordinary linoleic acid has double bonds at carbon atoms 9 and 12. Examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid and 10,12 octadecadienoic acid, 11,13 octadecadienoic acid. As used herein, “CLA” encompasses a single isomer, a selected mixture of two or more isomers, and a non-selected mixture of isomers obtained from natural sources, as well as synthetic and semisynthetic CLA. Examples of conjugated octadecatrienoic acids include the above CLA isomers that are desaturated at position 6.

As used herein, the term “isomerized conjugated linoleic acid” refers to CLA synthesized by chemical methods (e.g., aqueous alkali isomerization, non-aqueous alkali isomerization, or alkali alcoholate isomerization).

As used herein, the term “conjugated octadecatrienoic acid moiety” refers to any compound or plurality of compounds containing conjugated octadecatrienoic acids or derivatives. Examples include, but are not limited to fatty acids, alkyl esters, and triglycerides of conjugated octadecatrienoic acid.

As used herein, it is intended that “triglycerides” of CLA or conjugated octadecatrienoic acid contain CLA or conjugated octadecatrienoic acid at any or all of three positions (e.g., SN-1, SN-2, or SN-3 positions) on the triglyceride backbone. Accordingly, a triglyceride containing CLA or conjugated octadecatrienoic acid may contain any of the positional and geometric isomers of CLA or conjugated octadecatrienoic acid.

As used herein, it is intended that “esters” of CLA or octadecatrienoic acid include any and all positional and geometric isomers of CLA or conjugated octadecatrienoic acid bound through an ester linkage to an alcohol or any other chemical group, including, but not limited to physiologically acceptable, naturally occurring alcohols (e.g., methanol, ethanol, propanol). Therefore, an ester of CLA or esterified CLA or octadecatrienoic acid may contain any of the positional and geometric isomers of CLA or octadecatrienoic acid.

It is intended that “non-naturally occurring isomers” of CLA include, but are not limited to c11,t13; t11,c13; t11,t13; c11,c13; c8,t10; t8,c10; t8,t10; c8,c10; and trans-trans isomers of octadecadienoic acid, and does not include t10,c12 and c9,t11 isomers of octadecadienoic acid. “Non-naturally occurring isomers” may also be referred to as “minor isomers” of CLA as these isomers are generally produced in low amounts when CLA is synthesized by alkali isomerization.

As used herein, “c” encompasses a chemical bond in the cis orientation, and “t” refers to a chemical bond in the trans orientation. If a positional isomer of CLA or octadecatrienoic acid is designated without a “c” or a “t”, then that designation includes all possible positional isomers. For example, 10,12 octadecadienoic acid encompasses c10,t12; t10,c12; t10,t12; and c10,c12 octadecadienoic acid, while t10,c12 octadecadienoic acid or CLA refers to just the single isomer.

As used herein, the term “oil” refers to a free flowing liquid containing long chain fatty acids (e.g., CLA or conjugated octadecatrienoic acid), triglycerides, or other long chain hydrocarbon groups. The long chain fatty acids, include, but are not limited to the various isomers of CLA.

As used herein, the term “physiologically acceptable carrier” refers to any carrier or excipient commonly used with oily pharmaceuticals. Such carriers or excipients include, but are not limited to, oils, starch, sucrose and lactose.

As used herein, the term “oral delivery vehicle” refers to any means of delivering a pharmaceutical (e.g., a conjugated octadecatrienoic acid moiety) orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “volatile organic compound” refers to any carbon-containing compound which exists partially or completely in a gaseous state at a given temperature. Volatile organic compounds may be formed from the oxidation of an organic compound (e.g., CLA). Volatile organic compounds include, but are not limited to pentane, hexane, heptane, 2-butenal, ethanol, 3-methyl butanal, 4-methyl pentanone, hexanal, heptanal, 2-pentyl furan, octanal.

As used herein, the term “metal oxidant chelator” refers to any antioxidant that chelates metals. Examples include, but are not limited to lecithin and citric acid esters.

As used herein, the term “alcoholate catalyst” refers to alkali metal compounds of any monohydric alcohol, including, but not limited to, potassium methylate and potassium ethylate.

### DETAILED DESCRIPTION OF THE INVENTION

The present invention relates to a novel method of making certain compounds (especially fatty acids and derivatives thereof) being desaturated at a 6th carbon atom in a chain of carbon atoms, relative to the starting substrate; certain novel compounds being unsaturated at a 6th carbon atom in a chain of carbon atoms; and to compositions for nutritional and/or pharmaceutical use, comprising certain fatty acid compounds and derivatives thereof. The invention also provides for use of certain compounds as nutritional supplements and/or pharmaceuticals; and a method of making a nutritional and/or pharmaceutical composition. The detailed description is organized in the following sections: I. Production of Conjugated Octadecatrienoic Acid Moieties and Derivatives Thereof; II. Octadecatrienoic Acid Moieties and Derivatives Thereof; and III. Uses of Octadecatrienoic Acid Moieties and Derivatives Thereof.

I. Production of Conjugated Octadecatrienoic Acid Moieties and Derivatives Thereof

In some embodiments, the present invention provides methods of producing a molecule which, relative to a substrate, is desaturated at the 6th carbon atom in a chain of carbon atoms, the method comprising the steps of: contacting the substrate with a Δ6 desaturase enzyme obtainable from Spirulina spp. in suitable conditions so as to cause formation of a product being desaturated at the 6th carbon atom; and collecting the product.

In preferred embodiments, the invention provides methods of converting a substrate of general formula (I)

Insert formula

to a product of general formula (II)

Insert formula

the method comprising the steps of: contacting the substrate with a Δ6 desaturase enzyme obtainable from Spirulina spp. in suitable conditions so as to cause formation of the product; and collecting the product; wherein R₁ is a C5 linear chain (preferably alkyl or alkenyl), substituted or unsubstituted, (preferably unbranched), and wherein R₂ is a C1-C20 linear chain (preferably alkyl or alkenyl), substituted or unsubstituted, and wherein R₃ and R₄ are, independently, H, OH, halide or methyl. Preferably R₂ is C8-C14, most preferably C12, and is typically mono- or (preferably) diunsaturated. Preferably at least one of R₃ and R₄ is H, most preferably both.

In preferred embodiments the substrate is an unsaturated fatty acid and/or comprises a trans carbon/carbon double bond. In other preferred embodiments, the substrate comprises an alkyl moiety (branched or, preferably, unbranched), preferably an unsaturated fatty acid, and preferably an unsaturated fatty acid comprising a trans carbon/carbon double bond. In particular the preferred substrate is a di-unsaturated fatty acid (being saturated at the 6th carbon atom) and the preferred product is the corresponding tri-unsaturated fatty acid, (being desaturated at the 6th carbon atom). The substrate is preferably a C14-C20 compound, most preferably a C18 compound, such as CLA. Preferred products are tri-unsaturated fatty acids derived from conjugated linoleic acid substrates, (i.e., conjugated octadecatrienoic acid moieties). Examples of preferred products include 6-c, 9-c, 11-t octadecatrienoic acid and 6-c, 10-t, 12-c octadecatrienoic acid.

Those skilled in the art will be well acquainted with the convention for numbering of carbon atoms in a molecule comprising a chain of linked carbon atoms. In general, where a carbon chain possesses a functional group at one end (e.g., a terminal hydroxyl group, as in alkanols), then the carbon atom to which that functional group is attached is considered position 1. Thus, for example, oleic acid has a carbon/carbon double bond between the ninth and tenth carbon atoms (counting the first carbon atom as that present in the carboxyl group), and may be represented as C_{18.1}^{Δ9} (i.e., has a single carbon/carbon double bond and is desaturated at the ninth carbon atom, in an 18 carbon atom chain).

In other embodiments, the substrate is crepenynic acid (or octadeca-9-en-12-ynoic acid), which via the method of the invention can be used to produce a corresponding Δ6 desaturated product (i.e., dehydrocrepenynic acid, or octadeca-6, 9-dien-12-ynoic acid).

The Δ6 desaturase enzyme of use in the invention is typically that which is obtainable from Spirulina platensis or Arthrospira platensis, in particular from a strain deposited in the Paris Culture Collection (S. platensis PCC8005). Other strains of Spirulina/Arthrospira from which the Δ6 desaturase enzyme may be obtained include PCC 6313, PCC 7345 and PCC 7939.

Those skilled in the technique of “biotransformations” will appreciate that, in the invention defined above, the step of contacting the substrate with the Δ6 desaturase need not involve any kind of isolation, extraction or purification of the enzyme. Thus, while it is possible (if desired) to prepare a Δ6 desaturase enzyme-containing extract from Spirulina, it is more convenient to contact the substrate with a plurality of cells of Spirulina organisms (such as S. platensis), which may generally be referred to as a “culture”, regardless of whether the cells are actively growing. Indeed, provided that the cells have produced the Δ6 desaturase, it is possible that the cells may be killed (e.g., by the action of a biocide or by sonication) and still be useful in the method of the invention, provided that the culture retains Δ6 desaturase activity. References to contacting the substrate with the Δ6 desaturase should therefore be construed, where the context permits, as encompassing contacting the substrate with a plurality of Spirulina cells comprising the Δ6 desaturase.

Methods of culturing cyanobacteria, such as Spirulina, are well known to those skilled in the art, and do not require any further elaboration. Conditions suitable for effecting the method of the invention will be apparent to the person skilled in the art, given the benefit of the present disclosure. Typically, the substrate will be contacted with the Δ6 desaturase enzyme at atmospheric pressure and at a temperature in the range 10-40° C., preferably 15-35° C. The substrate concentration may conveniently be in the range 0.1mM to 0.1M. The cells of Spirulina (if present) may be used free in suspension in a suitable liquid medium (which may be any medium suitable for the purpose, such as a phosphate buffered saline, or a cyanobacterial growth medium such as Zabrouk's medium), or else may be immobilized in some way (e.g., on an inert solid support, such as a filter, gel, mesh or the like).

The inventors have found that some substrates, which it is desired to desaturate at the Δ6 position, exert a bactericidal or bacteriostatic toxic effect on Spirulina. Accordingly, in a preferred embodiment, a culture of Spirulina organisms is grown up and the substrate is not *introduced into the culture in appreciable amounts until growth of the culture is substantially complete (i.e., at or near the point at which there is a maximum number of viable Spirulina cells in the culture, which may readily be determined by conducting a growth curve analysis of the culture under the conditions in question).

For the sake of simplicity, the substrate may be contacted with a Δ6 desaturase-containing culture of Spirulina as a bolus. However, the inventors consider that higher yields are obtainable if the substrate is introduced over a period of time (e.g., continuously at a low rate, or as a repeated number of introductions of substrate), say 24 hours.

The optimum conditions to be employed will depend on the identity of the substrate and preferred product, the identity of the Spirulina organism used (if any) and so on. The optimum conditions for any one set of circumstances can be readily ascertained by the person skilled in the art using routine trial-and-error.

The substrate may be contacted with the Δ6 desaturase for a time of variable duration, depending on circumstances. Typically the substrate may be incubated with the enzyme for at least 12 hours, preferably at least 24 hours. The yield of product typically starts to reach a plateau after 48-72 hours' incubation, and further contact with the enzyme does not greatly increase yield, although the rate of reaction will of course depend to some extent on the reaction conditions.

If desired, the product may be recovered in crude form, by recovering the culture medium and/or the Spirulina cells (if present). More preferably, the product is subjected to processing, so as to purify the product. Typically, the Δ6 desaturated compound will, if contacted with whole cells of an organism (such as Spirulina), become esterified (see Quoc et al, 1993 Biochim. Biophys. Acta 1168, 94-99), so as to form a lipid. Accordingly, the cells may be harvested from the culture by standard methods (e.g., centrifugation or filtration), and the cells containing the desired product collected. If it is desired to obtain the Δ6 desaturated compounds as free fatty acids, the lipid content of the collected cells and/or medium, if desired can be isolated (e.g., by extraction with an organic solvent such as isopropanol, and saponified by reaction with ethanolic potassium hydroxide. Alternatively the Δ6 desaturated compounds may be transesterified, e.g., to produce methyl esters by reaction with sodium methoxide in anhydrous methanol, allowing individual molecular species to be isolated (e.g., by chromatography). Such methods are well known to those skilled in the art (e.g., see Christie Gas Chromatography and Lipids, A Practical Guide, Oily Press, Dundee (1989).

It is contemplated that other Δ6 desaturases will find use in the methods of the present invention. Indeed, Δ6 desaturases from several organisms have been described (e.g., Mortiella alpina (U.S. Pat. No. 6,075,183, incorporated herein by reference); borage (PCT WO 96/21022, incorporated herein by reference; and Synechocystis (U.S. Pat. Nos. 5,552,306 and 5,614,393, each of which is herein incorporated by reference. These Δ6 desaturases can be screened for activity on conjugated substrates as described above.

It is also contemplated that purified Δ6 desaturases from the above sources may be utilized in the methods of the present invention. In some embodiments, the purified Δ6 desaturases are used in a suspension with the conjugated linoleic substrate. In other embodiments, the purified desaturases are immobilized on solid substrates such as porous chitosan, anion exchange resins, phenolic adsorbent resins, cation exchange resins, acrylic adsorbent resins, and hydrophobic polymers (See, e.g., U.S. Pat. Nos. 5,445,955 and 5,108,916, each of which is incorporated herein by reference).

The purified Δ6 desaturases may be provided from a variety of sources. For example, where the gene for the Δ6 desaturase is cloned (e.g., Δ6 desaturases from borage, Mortiella alpina, and Synechocystis), the Δ6 desaturase may be produced by known molecular biology techniques (e.g., expression in host cells). The Δ6 desaturase can be isolated from host cells or native sources by column chromatography. Methods for recovering and purifying Δ6 desaturase from host cells or native sources include, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein refolding steps can be used as necessary, in completing configuration of the mature protein. In still other embodiments of the present invention, high performance liquid chromatography (HPLC) can be employed for final purification steps. It is contemplated that column fractions containing Δ6 desaturase activity may be identified by a biological assay wherein an aliquot of the fraction is added to a reaction containing conjugated linoleic acid. Samples containing a Δ6 desaturase are identified by MS/GC chromatography as described in the examples. As will be recognized, a number of columns can be utilized for purification by the following the activity with the biological assay.

The conjugated linoleic acid starting material may be produced by a variety of methods. In general, the preferred starting materials for conjugation with alcoholate catalysts are sunflower oil, safflower oil, and corn oil. In some embodiments of the present invention, the conjugated linoleic acid is produced by nonaqueous alkali isomerization. The reaction conditions of the controlled isomerization process allow for precise control of the temperature (and constant ambient pressure) of the conjugation process. Preferably the alkali is an inorganic alkali such as potassium hydroxide, cesium hydroxide, cesium carbonate or an organic alkali such as tetraethyl ammonium hydroxide. The catalyst is preferably provided in a molar excess as compared to the fatty acid content of oil. The solvent is propylene glycol. Preferably, the reaction is conducted within a temperature range 130 to 165° C., most preferably at about 150° C. The time of the reaction may vary, however, there is an increased likelihood of the formation of undesirable isomers when the reaction is conducted for long periods of time. A relatively short reaction time of 2.0 to 6.5 hours has proved satisfactory for excellent yields.

It will be understood to a person skilled in the art that to produce the desired composition, the reaction conditions described above may be varied depending upon the oil to be conjugated, the source of alkali, and equipment. Preanalysis of a particular oil may indicate that the conditions must be varied to obtain the desired composition. Therefore, the temperature range, pressure, and other reaction parameters represent a starting point for design of the individual process and are intended as a guide only. For example, it is not implied that the described temperature range is the only range which may be used. The essential aspect is to provide precise temperature control. However, care must be taken because increasing the pressure may lead to less than complete isomerization and the formation of undesirable isomers. Finally, the length of the conjugation reaction may be varied. Generally, increasing amounts of undesirable isomers are formed with increasing length or reaction time. Therefore, the optimal reaction time allows the reaction to go nearly or essentially to completion but does not result in the formation of undesirable isomers.

Following the conjugation reaction, the resulting CLA containing composition may be further purified. To separate the fatty acids from the conjugation reaction mix, the reaction mix is cooled to approximately 95° C., an excess of water at 50° C. is added, and the mixture slowly stirred while the temperature is reduced to about 50° C. to 60° C. Upon addition of the water, a soap of the fatty acids is formed and glycerol is formed as a by-product. Next, a molar excess of concentrated HCl is added while stirring. The aqueous and nonaqueous layers are then allowed to separate at about 80-90° C. The bottom layer containing water and propylene glycol is then drawn off. The remaining propylene glycol is removed by vacuum dehydration at 60-80° C.

The dried CLA composition may then preferably be degassed in degassing unit with a cold trap to remove any residual propylene glycol. Next, the CLA is distilled at 190° C. in a molecular distillation plant at a vacuum of 10⁻¹ to 10⁻² millibar. The advantage of this purification system is the short time (less than one minute) at which the CLA is held at an elevated temperature. Conventional batch distillation procedures are to be strictly avoided since they involve an elevated temperature of approximately 180-200° C. for up to several hours. At these elevated temperatures the formation of undesirable trans-trans isomers will occur. Approximately 90% of the feed material is recovered as a slightly yellow distillate. The CLA may then be deodorized by heating to about 120°-170° C., preferably at about 150° C. for 2 hours to improve smell and taste. Excessive heat may result in the formation of trans-trans isomers. These procedures produce a CLA composition with a solvent level of less than about 5 ppm, preferably less than about 1 ppm. This process eliminates toxic trace levels of solvent so that the resulting composition is essentially free of toxic solvent residues.

It is contemplated that the above conjugation methods may also be utilized to conjugate γ-linolenic acid to produce c6,t10,c12 octadecatrienoic acid. In some preferred embodiments, the starting material borage oil, which contains a high concentration of γ-linolenic acid.

In other embodiments, the present invention also provides methods for producing alkyl esters of conjugated octadecatrienoic acid. After fat splitting and dehydration, the free fatty acids are combined with methanol or another monohydric low molecular weight alcohol and heated to the temperature at which the alcohol boils. Esterification proceeds under refluxing conditions with removal of the reaction water through a condenser. After the addition of a further quantity of the same or a different monohydric alcohol an alcoholate catalyst is blended into the ester mix (See, e.g., U.S. Pat. No. 3,162,658, incorporated herein by reference). Typical alcoholate catalysts are sodium or potassium ethoxide, or their methyl, butyl, or propyl counterparts.

In the esterification, methanol or ethanol are preferred, although other branched or straight chain monohydric alcohols may be used. The longer the aliphatic chain of the alkyl group, the more lipid compatible the material becomes. Also the viscosity tends to increase. For different types of feed or food, whose consistency varies, product of varying viscosity can be used to obtain the desired flow or compounding characteristics without affecting the therapeutic or nutritional properties arising from the CLA moieties. The theory and practice of esterification are conventional. A basic explanation of the most common methods is set forth in the McGraw-Hill Encyclopedia of Science & Technology, McGraw-Hill Book Co., N.Y.: 1996 (5th ed.).

In the isomerization step, it has been found that alcoholate catalysis produced a much superior product than aqueous alkali mediated isomerization. The latter process always produced undesirable isomers even under mild reaction conditions. The milder conditions do give lower amounts of unwanted isomers, but at the great expense of yield, as shown in the Examples. In most systems the appearance of the c9,t11 and t10,c12 isomers dominates and they are formed in roughly equimolar amounts. It has not heretofore been possible to control the isomerization of the one isomer to the exclusion of the other. While it is desirable to increase the percentage of one or the other isomer (depending on the physiological effect to be achieved), at present this must largely be carried out by adding an enriched source of the *desired isomer.

In some embodiments, it is further contemplated that glycerol and esters of glycerol should be removed before making monoesters of fatty acids. Traces of glycerol present during conjugation contribute to the production of trimethoxypropane and triethoxypropane. Therefore, prior to conjugation, it is preferable to distill monoesters obtained by alcoholysis.

In some embodiments, purified isomers of CLA are utilized as the starting material for the production conjugated octadecatrienoic acid isomers. For example, relatively pure t10,c12 CLA may be provided by the method of Scholfield and Koritalia, JOACS 47(8):303 (1970) or Berdeau et al., JAOCS 75:1749-1755 (1998). Additionally, pure isomers may be isolated by preparative scale chromatography. Such isomers are commercially available from vendors such as Matreya, State College, Pa.

In other embodiments, purified isomers are provided by treating CLA isomers under conditions that cause migration of the double bond system. In preferred embodiments, the conditions comprise heating at least one isomer to about 200-240° C., preferably to about 220° C. In other embodiments, the conditions further comprise reacting the partially purified or concentrated isomer or isomers under nitrogen in a sealed container. Referring to Table 1, the preparations of isomers in column 1 can be used to produce preparations containing a substantial amount of the corresponding isomer in column 2. After the initial conversion reaction, the preparation will contain both the starting isomer and the “sister” isomer. Likewise, the preparations of isomers in column 2 can be used to produce substantial amounts of the corresponding isomer in column 1. The preparations containing both isomers may be further treated to purify the sister isomer (e.g., by gas chromatography). As will be understood by those skilled in the art, it is possible to start with more than one partially purified isomer, thereby producing a preparation containing four, six, eight or more isomers. In further embodiments, a purified preparation of the sister isomer may be prepared by methods known in the art (i.e., gas-liquid chromatography) from the treated preparation containing the initial isomer and its sister isomer.

TABLE 1

| Column 1 | Column 2 |
|---|---|
| c9,t11 | t8,c10 |
| t10,c12 | c11,t13 |
| c7,t9 | t6,c8 |
| t11,c13 | c12,t14 |
| c6,t8 | t5,c6 |
| c5,t7 | t4,c6 |
| c4,t6 | t3,c5 |
| t12,c14 | c13,t15 |
| t13,c15 | c14,t16 |

II. Octadecatrienoic Acid Moieties and Derivatives Thereof

The methods defined above are useful for producing certain compounds which are believed to be novel per se and which possess various useful qualities. Thus, in some embodiments, the invention provides c-6, c-9, t-11 octadecatrienoic acid or a derivative thereof (i.e., a conjugated octadecatrienoic acid moiety). In other embodiments, the invention provides c-6, t-10, c-12 octadecatrienoic acid or a derivative thereof (i.e., a conjugated octadecatrienoic acid moiety). Those skilled in the art will appreciate that the carboxyl group in these molecules is weakly acidic, so that salts of the compounds may readily be formed, and such salts and other derivatives are considered to fall within the scope of the invention. The cations of the salts may be any convenient cation and include, for example, ammonium (NH₄⁺), sodium, potassium or magnesium ions. Other derivatives included within the scope of the invention include substituted compounds and esters formed at the carboxyl group. Such esters include compounds formed by condensation reactions between the carboxyl group of the respective fatty acids and the hydroxyl groups of alkyl, alkenyl or aromatic compounds (substituted or unsubstituted). Thus for example, the invention also provides complex lipids formed by esterification of the carboxyl groups, especially glycerides such as phosphoglycerides or triacylglycerides. In still other embodiments, the invention provides octadeca-6, 9-dien-12-ynoic acid (especially c-6, c-9 octadeca-6; 9-dien-12-ynoic acid) or a derivative thereof (such as a salt, ester and the like, as explained above).

In some embodiments, the invention provides compounds of the formula:

R₁—O—R₂

wherein R₁ is a conjugated octadecatrienyl group (e.g., c-6, c-9, t-11 octadecatrienoyl, c-6, c-9, c-11 octadecatrienoyl, c-6, t-9, t-11 octadecatrienoyl, c-6, t-9, c-11 octadecatrienoyl, c-6, t-10, c-12, octadecatrienoyl, c-6, c-10, t-12, octadecatrienoyl, c-6, t-10, t-12, octadecatrienoyl, and c-6, c-10, c-12, octadecatrienoyl) and R₂ is selected from the group consisting of hydrogen, alkyl groups (e.g., methyl, ethyl, or propyl), glycerol, and phosphoglycerol.

As described above, the present invention contemplates the use of derivatives of octadecatrienoic acids. For example, the octadecatrienoic acid may be free or bound through ester linkages as described above or provided in the form of an oil containing octadecatrienoic acid triglycerides. In these embodiments, the triglycerides may be partially or wholly comprised of CLA attached to a glycerol backbone. The CLA may also preferably be provided as a methylester or ethylester. Furthermore, the CLA may be in the form of a non-toxic salt, such as a potassium or sodium salt (e.g., a salt formed by reacting chemically equivalent amounts of the free acids with an alkali hydroxide at a pH of about 8 to 9).

In one embodiment of the present invention, a novel triacylglycerol is synthesized from the novel octadecatrienoic acid isomer or mixture of isomers disclosed above. The triacylglycerols highly enriched for octadecatrienoic acid (e.g., 90-96 percent) may be confirmed by H NMR. Esterification proceeds using immobilized Candida antarctica Lipase. Preferably, the octadecatrienoic acid-containing triglyceride will contain at least 40 and upwardly 50 percent of c6,c9,t11-octadecatrienoic and c6,t10,c12-octadecatrienoic acids, and mixtures thereof. In some embodiments, there will be less than one percent of 6,8,10 and 6,11,13 isomers or less than five percent in the aggregate. In some embodiments, the resultant triacylglycerol is not purified further to remove all levels of phosphatidyl and sterol residues. In any event, those levels remaining will be adequate for commercial applications involving safe, edible products in feed and food. In other embodiments, the triacylglycerol is further purified by molecular distillation.

The immobilized Candida antarctica lipase is to be employed in a manner similar to that described for n-3 type polyunsaturated fatty acids (See, e.g., Harraldson et al. Acta Chemica Scandinavica 45:723-730 (1991); Haraldsson et al., Tetrahedron 51:941-952 (1995); Haraldsson et al., JAOCS 74:1419-1424 (1997)). The esterification reaction is conducted at 50°-75° C., preferably 65° C., in the absence of any solvent and a vacuum employed in order to remove the co-produced water or alcohols (from esters) upon formation. This shifts the triacylglycerol production to completion and ensures a highly pure product virtually free of any mono- and diacylglycerols in essentially quantitative yields. Stoichiometric amounts of free fatty acids may be used, i.e. 3 molar equivalents as based on glycerol or 1 molar equivalent as based on number of mol equivalents of hydroxyl groups present in the glycerol moiety. Only 10% dosage of lipase as based on total weight of substrates is needed, which can be used a number of times. This is very important from the productivity point of view. All this, together with the fact that no solvent is required, renders this process a high feasibility from the scaling-up and industrialization point of view, since the cut in volume and bulkiness is enormous. Also, a slight excess (<5/5) of free fatty acids may be used in order to speed up the reaction toward the end and ensure a completion of the reaction.

At the initiation of the reaction, the 1- or 3-mono-acylglyeride is formed first, followed by the 1,3 diacylglyeride, and finally the triglyceride at the more extended reaction times. The mono- and diacylglyerides are useful intermediates in that they manifest biological activity, but have greater solubility in aqueous cellular environments and can participate in alternative molecular synthetic pathways such as synthesis of phospholipids or other functional lipids. In contrast, triglycerides are frequently deposited intact in cell membranes or storage vesicles. Thus, the administration of octadecatrienoic acid in mono-, di- or triglycerol form rather than free fatty acid or ester, may influence the mode and distribution of uptake, metabolic rate and structural or physiological role of the octadecatrienoic acid component.

The compounds described above may be provided in substantially pure form, or at least partly purified (e.g., typically present in an amount of at least 0.001% w/w, preferably at least 0.01% w/w, more preferably at least 0.1% w/w and most preferably at least 1% w/w, in a composition containing the compounds). They may additionally be provided in a form suitable for administration to a mammalian subject, typically a human.

In other embodiments, the octadecatrienoic acid moiety is formulated as a powder. This example describes the production of a powder containing CLA triglycerides. In some embodiments, warm water (about 538.2 ml at 110-120° F.) and an excipient (e.g., HI-CAP 100 (National Starch, Bridgewater, N.J.)) are combined and agitated until the dispersion is free of any lumps. A triglyceride containing octadecatrienoic acid (about 230.9 g) is then added and the mixture homogenized for 2 min in a lab homogenizer (e.g., Arde Berinco at setting 30). The pre-emulsion is then homogenized at full speed for 2-5 min (one pass at 3500 psi total pressure). The particle size is checked and should be from about 0.8 to 1.0 microns. The emulsion is then spray dried in a seven foot conical dryer at the following settings: inlet temperature (190-215° C.); outlet temperature (95-100° C.). Outlet temperature is maintained by adjusting the emulsion feed rate.

Furthermore, it is also advantageous to add compounds to conjugated octadecatrienoic moiety preparations to decrease oxidation during storage. Compounds that prevent oxidation (antioxidants) have two general mechanisms of action. The first is the prevention of oxidation by lipid peroxide radical scavenging. Examples include but are not limited to tocopherols and ascorbylpalmitate. The second mechanism for preventing oxidation is by the chelation of metal ions. Examples of metal oxidant chelators include, but are not limited to, citric acid esters and lecithin. Some commercially available compounds (e.g., Controx, Grunau (Henkel), Illertissen, DE) include both peroxide scavengers and metal chelators (e.g., lecithin, tocopherols, ascorbylpalmitate, and citric acid esters). In some embodiments of the present invention, metal oxidant chelators are added to conjugated octadecatrienoic moiety containing compounds to prevent oxidation. In other embodiments, a combination of metal oxidant chelators and peroxide scavengers is included in the CLA composition.

In some embodiments, gas chromatography/mass spectroscopy is used to detect the presence of volatile organic breakdown products of conjugated octadecatrienoic moieties. In other embodiments, oil stability index (OSI) measurements are used to detect the presence of volatile organic breakdown products of conjugated octadecatrienoic moieties. In some embodiments of the present invention methods for the removal of pro-oxidants (e.g., iron) from conjugated octadecatrienoic moiety containing samples are provided. Methods include, but are not limited to, distillation and adsorption.

In preferred embodiments, precautions are taken during purification to prevent oxidation during storage. These precautions include the removal of compounds that serve as pro-oxidants, including but not limited to iron or other metals. In some embodiments, metals are removed by treating with adsorbing agents, including but not limited to bleaching earth, active charcoal zeolites, and silica. In other embodiments, the pro-oxidants are removed by distillation.

In other embodiments, pro-oxidants are removed in a distillation process. For example, distillation of a triacylglyceride of CLA is preferably performed on a molecular distillation apparatus. Distillation is carried out at 150° C. and a pressure of 10⁻² mbar. The present invention is not intended to be limited to the conditions described for distillation. Other temperatures and pressures are within the scope of the present invention.

As described above, in some embodiments, oxidation of conjugated octadecatrienoic moieties is prevented by the addition of metal oxidant chelators or peroxide scavengers to the finished product. In some embodiments, the amount of oxidation is measured by the oil stability index (OSI). The OSI (See e.g., AOCS official method Cd 12b-92) is a measurement of an oil's resistance to oxidation. It is defined mathematically as the time of maximum change of the rate of oxidation. This rate can be determined mathematically. Experimentally, the OSI is calculated by measuring the change in conductivity of deionized water is which volatile organic acids (oxidation products) are dissolved. When performing OSI measurements, it is important to avoid contamination by trace amounts of metals, which can accelerate the oxidation process. This is generally accomplished by careful washing of all glassware used with a cleaning solution lacking chromate or surfactants. Water must be deionized and all solvents must be of a highly purified grade.

Accordingly, in some embodiments, preparations or compositions containing conjugated octadecatrienoic moieties contain less than 500 ppm volatile organic compounds (e.g., preferably less than 100 ppm volatile organic compounds, more preferably less than 50 ppm volatile organic compounds, and most preferably less than 10 ppm volatile organic compounds).

III. Uses of Octadecatrienoic Acid Moieties and Derivatives Thereof

The compounds described above find a number of possible uses. They are useful in themselves as nutritional supplements and as intermediates in the production of other products, for example as nutritional supplements. The present invention is not limited to a particular mechanism of action. Indeed, an understanding of the mechanism of action is not necessary to practice the present invention. Nevertheless, it is believed that conjugated octadecatrienoic acid moieties (e.g., c-6, c-9, t-11 and c-6, t-10, c-12 octadecatrienoic acids and triglycerides containing these fatty acids) are useful as precursors of arachidonic acid (c-5, c-8, c-11, c-14 eicosatetraenoic acid) analogues. Following consumption by a mammalian subject, the compounds may be further desaturated and chain-extended, so as to form (C_{20:4}) analogues of arachidonic acid (c-5, c-8, c-11, t-13 and c-5, c-8, t-12, c-14 eicosatetraenoic acids respectively), which may then act as inhibitors of eicosenoid metabolism. The arachidonic acid analogues are believed to possess a number of therapeutic effects, especially in: preventing and/or reducing atherosclerosis; preventing or treating undesirable proliferation of cells in neoplastic conditions; increasing the efficacy of the immune system; and promoting deposition of protein in the body in preference to the deposition of fat. Δ6 unsaturated fatty acids and derivatives thereof are predicted by the inventors to exhibit more favorable pharmacodynamics and/or greater biological activity in vivo than the equivalent compounds saturated at position 6, as desaturation at position 6 is generally thought to occur only very slowly in mammalian tissues.

Thus, in some embodiments, the present invention provides, a pharmaceutical or nutritional composition comprising a Δ6 unsaturated fatty acid moiety, especially a c-6, c-9, t-11 and/or c-6, t-10, c-12 octadecatrienoic acid moiety (which term should be construed as including salts or other physiologically acceptable derivatives especially glycerides or other esters), together with a physiologically acceptable excipient, bulking agent or diluent. The composition may be administered in liquid form or as a solid. Liquid compositions may be injected (e.g., sub-cutaneously, intra-venously or intra-muscularly) or consumed orally. Solid compositions may take the form of tablets, capsules and the like. Suitable excipients, bulking agents or diluents will readily be apparent to those skilled in the art and include, for example, carbonates (especially calcium carbonate), silicates, water, aqueous solutions and buffers and so on. Solid compositions in the form of tablets or capsules are generally to be preferred for their ease of administration. When intended for use as a nutritional composition (to be consumed orally), it may be desirable for the composition to comprise one or more additional components, such as vitamins, minerals, flavorings, anti-oxidants, stabilizers, preservatives and the like.

An effective dose of the octadecatrienoic acid-containing composition will depend on the condition to be treated and the route of administration. As a guide, oral administration of between 10 mg and 10 grams per day will be likely to be an effective dose in preventing or treating atherosclerosis, with a preferred dose in the range 30 mg to 1 gm. The dose may conveniently be given in a single tablet, or given at intervals during a 24 hour period (say, 100 mg at approximately 8 hr intervals).

In still further embodiments, the invention provides a method of making a composition for consumption by a mammalian (preferably human) subject; the method comprising the step of providing a Δ6 unsaturated fatty acid moiety, especially a c-6, c-9, t-11 and/or c-6, t-10, c-12 octadecatrienoic acid moiety (which terms should be construed as including salts or other physiologically acceptable derivatives of the acid, such as glycerides or other esters); mixing said acid with a suitable excipient, bulking agent or diluent; and packaging the resulting mixture (preferably in unitary dose form).

In other embodiments, the invention provides for use of Δ6 unsaturated fatty acid moiety, especially a c-6, c-9, t-11 and/or c-6, t-10, c-12 octadecatrienoic acid moiety (which includes salts or other physiologically acceptable derivatives such as glycerides or other esters) in the manufacture of a medicament or nutritional supplement. In particular, the medicament or nutritional supplement may be administered to a mammalian (especially a human) subject, in order to affect eicosenoid metabolism in the subject. For example, the medicament may be administered to act as an arachidonic acid analogue, with beneficial effect in certain conditions.

It will be appreciated by those skilled in the art that the c-6, c-9, t-11 and/or c-6, t-10, c-12 octadecatrienoic acids may not, indeed probably will not, have a direct effect per se on eicosenoid metabolism, but following administration to the subject will be converted within the subject into other compounds which will exert an effect on eicosenoid metabolism.

In some embodiments, the invention provides for a method of forming c-5, c-8, c-11, t-13 and/or c-5, c-8, t-12, c-14 eicosatetraenoic acid in a mammalian subject, the method comprising the steps of providing c-6, c-9, t-11 and/or c-6, t-10, c-12 octadecatrienoic acid (respectively) (or a salt or other physiologically acceptable derivative such as glycerides or other esters); and administering said acid(s) to the subject; such that the octadecatrienoic acid(s)or physiologically acceptable derivative thereof is desaturated and chain-extended to form the corresponding eicosatetraenoic acid.

In still other embodiments, the invention provides a method of inhibiting arachidonic acid in a mammalian subject, the method comprising providing c-6, c-9, t-11 and/or c-6, t-10, c-12 octadecatrienoic acid (or salt or other physiologically acceptable derivative thereof); and administering an effective dose of the acid(s) to the subject. In further aspects the invention provides a method of causing one or more of the following in a mammalian subject; preventing and/or treating atherosclerosis; preventing and/or treating cancer; promoting deposition of protein; preventing or reducing deposition of fat; and increasing the efficacy of the immune system; the method comprising the administration of an effective dose of c-6, c-9, t-11 and/or c-6, t-10, c-12 octadecatrienoic acid (which term encompasses salts or other physiologically acceptable derivatives thereof).

The conjugated octadecatrienoic acids moieties of the present invention may be provided by any of a number of routes, including, but not limited to, oral, intravenous, intramuscular, intra-arterial, intramedullary, intrathecal, intraventricular, transdermal, subcutaneous, intraperitoneal, intranasal, enteral, topical, sublingual or rectal means. Further details on techniques for formulation for and administration may be found in the latest edition of Remington's Pharmaceutical Sciences (Maack Publishing Co., Easton, Pa.).

An effective amount of the conjugated octadecatrienoic acid moiety may also be provided as a supplement in various food products, including animal feeds, and drinks. For the purposes of this application, food products containing a octadecatrienoic acid moiety means any natural, processed, diet or non-diet food product to which exogenous octadecatrienoic acid moiety has been added. The conjugated octadecatrienoic acid moiety may be added in the form of free fatty acids, esters, or as an oil containing partial or whole triglycerides. Therefore, CLA may be directly incorporated into various prepared food products, including, but not limited to diet drinks, diet bars, supplements, prepared frozen meals, candy, snack products (e.g., chips), prepared meat products, milk, cheese, yogurt and any other fat or oil containing foods. For example, a liquid dietetic food for parenteral administration to humans containing emulsified fat particles of about 0.33-0.5 micrometers in diameter is disclosed. The emulsion contains 0.5 mg/gm to 10 mg/gm of conjugated octadecatrienoic acid or triglyceride or alternatively, 0.3% to 100% conjugated octadecatrienoic acid or triglyceride based on the food lipid or 0.03 gm to 0.3 gm conjugated octadecatrienoic acid or triglyceride per 100 calorie serving. This application also discloses a baby formula containing similar amounts of conjugated octadecatrienoic acid or triglyceride along with 2.66 gm of protein, 5.46 gm of fat, 10.1 gm of carbohydrate, 133 gm of water, and vitamins and minerals in RDA amounts. Another example of a low-residue liquid enteral dietetic product useful as a high-protein, vitamin and mineral supplement is disclosed. This supplement contains conjugated octadecatrienoic acid or triglyceride at 0.05% to about 5% by weight of the product, or by 0.3% to about 100% of the lipid present or about 0.03 to 0.3 gm conjugated octadecatrienoic acid or triglyceride per 100 calories. Additionally, 140 calories of a representative formula can contain 7.5 gm of egg white solids, 0.1 gm conjugated octadecatrienoic acid or triglyceride, 27.3 gm carbohydrate such as sucrose or hydrolyzed cornstarch, 1.9 gm of water, and vitamins and minerals in RDA amounts.

Other examples of foods, rations, and other products in which conjugated octadecatrienoic acid can be substituted for CLA are disclosed in U.S. Pat. Nos. 6,019,990; 6,042,132; 6,034,132; 6,015,833; and 6,060,519; each of which is incorporated herein by reference. In still further embodiments, the present invention provides octadecatrienoic acid compositions for oral administration to animals, including ruminants and non-ruminants such as pigs, cattle, horses, sheep, goats, dogs, and cats. In some embodiments, the compositions are feed supplements that contain an effective amount of a conjugated octadecatrienoic acid moiety. In other embodiments, the compositions are feeds that contain an effective amount of a conjugated octadecadienoic acid moiety. In some embodiments, the compositions are veterinary pharmaceuticals formulated with a suitable excipient or diluent.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); μM (micromolar); kg (kilograms); g (grams); mg (milligrams); μg (micrograms); ng (nanograms); L or 1 (liters); ml (milliliters); μl (microliters); cm (centimeters); mm (millimeters); nm (nanometers); ° C. (degrees centigrade); KOH (potassium hydroxide); HCL (hydrochloric acid); Hg (mercury).

1.1 Materials:

9-Cis,11-trans-(abbreviated as 9-c, 11-t) octadecadienoic acid (87% pure) was prepared synthetically, whilst 10-trans,12-cis(10-t, 12-c)-octadecadienoic acid was a gift from J. L. Sébédio (INRA, Dijon, France). Seed oils of Crepis alpina (rich in crepenynic acid) and Vernonia galamensis (rich in vemonic acid) were a gift from Richard Adlof (USDA Laboratory, Peoria, USA)

1.2 Incubations:

Spirulina platensis (Arthrospira sp.) PCC8005 was grown in Zabrouk's medium in 50 ml batch culture in 100 ml erlenmeyer flasks at 30° C. with illumination of 50 μmol photons m⁻² sec⁻². Cultures were harvested following 5 days growth by centrifugation at 40,000 g in a F0650 rotor in a Beckman Avanti centrifuge 20 minutes, cooled rotor @4° C. The algal pellet was carefully resuspended in fresh Zabrouk's medium under aseptic conditions.

Initial time-course experiments were carried out by resuspending the algal pellet in 50 ml fresh medium, which was then divided into 10 ml aliquots, each containing 0.5 ml of 1.8mM ammonium fatty acid substrate and incubating under the above conditions. Control cultures incubated without linoleate were also set up. The algal material was collected from a control culture and a culture incubated with a CLA isomer at 24 hour time points between T=0 and T=96 hours, by centrifugation as described above. The algal pellet was washed four times in 50 ml freshly drawn MilliQ water and then freeze dried prior to analyses.

In later experiments where bulk material was required for a more detailed analysis of the metabolite produced, 4×50 ml cultures were grown and harvested as above. The algal pellet from all four cultures was then resuspended in a total volume of 50 ml fresh Zabrouk's medium and incubated with 0.5 ml of 1.8 mM ammonium fatty acid substrate for 96 hours under the above conditions. Following this incubation the algal material was pelleted, washed and freeze dried as described above.

1.3 Extraction of Lipids:

Lipids were extracted from freeze-dried S. platensis with isopropanol as described previously (Nichols, Biochim. Biophys. Acta, 70: 417-422 (1963)).

1.4 Derivatization:

Lipid extracts were converted to methyl esters by base-catalysed transesterification (Christie, Gas Chromatography and Lipids. A Practical Guide, Oily Press, Dundee (1989)). A portion was hydrolysed with 0.1M 10% aqueous potassium hydroxide to the free acids (Christie, Gas Chromatography and Lipids. A Practical Guide, Oily Press, Dundee (1989)). The latter were converted to the picolinyl ester derivatives by the procedure of Balazy and Nies, Biomed. Environ. Mass Spectrom., 18, 328-336 (1989)., or to the 4,4-dimethyloxazoline derivatives by the method of Luthria & Sprecher, Lipids, 28, 561-564 (1993).

1.5 Gas Chromatography:

Gas chromatographic analyses were effected with a Hewlett Packard HP 5890 Series II (Hewlett Packard Ltd, Wokingham, UK) equipped with a splitless/split injector and a flame-ionization detector. The temperature of both the injector and detector was 250° C. Hydrogen was the carrier gas. The analyses were performed with a column (fused silica capillary) coated with carbowax (Chrompack UK Ltd, London, 30 m×30.25 mm i.d.). The oven temperature was programmed from 170 to 220° C. at 4° C./min.

1.6 High-Performance Liquid Chromatography (HPLC):

In order to obtain better quality mass spectra from minor components, the latter were concentrated in the form of methyl esters by reversed-phase HPLC (Christie, Lipids, 33: 343-353 (1998)). A Gynkotek model 480 HPLC pump was utilized with a column of Hichrom RPB™ (250×4.6 mm; Hichrom Ltd, Reading, UK) and acetonitrile as mobile phase, with the flow rate programmed from 0.5 to 1.5 mL/min over 30 min, and held at this for a further 5 min. The temperature of the column was maintained at 20° C. The sample (0.5 mg) was injected in a solution (10 μL) of acetone-acetonitrile (1:9,v/v). An evaporative light-scattering detector was used in test runs, but timed fractions were collected in micro-preparative applications in the absence of a detector. The metabolite of interest from the CLA incubations eluted with the triene fraction.

1.7 Silver Ion Chromatography:

Isolute™ SCX solid-phase extraction columns were obtained from Jones Chromatography (Hengoed, Mid Glamorgan, Wales). They comprise phenyl sulphonate groups bound to silica. The columns were converted to the silver ion form as described previously (Christie, J. Lipid Res., 30: 1471-1473 (1989)), the silver ions complexing with the carbon/carbon double bonds in lipids to allow separation. The triene fraction from the reversed-phase-HPLC column was fractionated with the, the metabolite of interest eluting with the fraction in which dienes are normally expected with acetone as mobile phase. Linolenate remained on the column under these conditions and did not interfere with subsequent GC-MS analyses.

1.8 Gas Chromatography-Mass Spectrometry (GC-MS):

The derivatives were submitted to GC-MS with a Hewlett Packard 5890 Series II plus gas chromatograph attached to an HP model 5989 MS Engine. The latter was used in the electron impact mode at 70 eV with a source temperature of 250° C. The GC was fitted with on-column injection. For picolinyl ester and DMOX derivatives (see “Results And Discussion” below), a capillary column of fused silica coated with Supelcowax 10™ (25 m×0.25 mm, 0.25 μm film; Supelco UK, Poole, UK) was used. After holding the temperature at 80° C. for 3 min, the oven temperature was increased by temperature-programming at 20° C./min to 180° C., then at 2° C./min to 280° C., where it was held for 15 min. Helium was the carrier gas at a constant flow-rate of 1 mL/min, maintained by electronic pressure control.

2) Results and Discussion

9-c, 11-t-octadecadienoic acid was incubated with S. platensis for various time periods to compare the fatty acid composition with that of controls, and the results are shown in Tables 1a and 1b.

Table 1a and b:

Fatty acid composition (wt %) of lipids extracted from S. platensis following incubation (controls or with added 9-cis,11-trans-octadecadienoic acid), together with the total weight of lipid in each extract μg).

TABLE 1a
Controls

| | | 0h | 24h | 48h | 72h | 96h |
|---|---|---|---|---|---|---|
| palmitic acid | 16:0 | 41.7 | 41.0 | 41.6 | n.d. | 40.4 |
| palmitoleic acid | 16:1 | 6.7 | 7.1 | 6.7 | n.d. | 7.1 |
| stearic acid | 18:0 | 2.3 | 1.7 | 1.6 | n.d. | 2.3 |
| oleic acid | 18:1 | 5.7 | 3.8 | 3.6 | n.d. | 4.9 |
| linoleic acid | 18:2 | 10.8 | 9.7 | 10.1 | n.d. | 9.0 |
| linolenic acid | | 32.8 | 36.8 | 36.4 | n.d. | 36.3 |
| 18:3 (n-6) | | | | | | |
| Amount μg | | 88.0 | 134.7 | 175.4 | n.d. | 214.3 |

TABLE 1a
CLA

| | | 0h | 24h | 48h | 72h | 96h |
|---|---|---|---|---|---|---|
| palmitic acid | 16:0 | 42.0 | 30.7 | 32.7 | 34.3 | 35.4 |
| palmitoleic acid | 16:1 | 6.5 | 4.7 | 5.3 | 5.4 | 5.7 |
| stearic acid | 18:0 | 2.6 | 0.9 | 1.2 | 1.7 | 1.9 |
| oleic acid | 18:1 | 4.9 | 4.0 | 8.6 | 15.4 | 21.6 |
| linoleic acid | 18:2 | 11.7 | 8.2 | 6.9 | 4.9 | 3.9 |
| linolenic acid | | 32.2 | 19.5 | 19.0 | 16.5 | 15.1 |
| 18:3 (n-6) | | | | | | |
| 9,11 - 18:2 | | 0.0 | 27.8 | 21.2 | 16.2 | 10.5 |
| 6,9,11 - 18:3 | | 0.0 | 4.1 | 5.1 | 5.7 | 5.8 |
| Amount μg | | 85.3 | 170.3 | 188.3 | 211.8 | 166.2 |

Table 1b shows that the Spirulina cells do not contain any 9-c, 11-t octadecadienoic acid at the start of the incubation, but rapidly take up the compound from the surrounding medium (27.8% of lipid at 24 hrs), whereafter the di-unsaturated compound is converted to the corresponding 6,9,11 tri-unsaturated fatty acid, with a plateau level attained after about 72 hours.

In the controls (Table 1a), the relative proportions of the various fatty acids, with palmitic and γ-linolenic acids as the main components, were constant; the absolute amount increased with time. The extracts incubated with the CLA isomer contained two new components, in comparison to the controls, the CLA isomer per se and a later running fatty acid (as determined by GC-gas chromatography), the retention time of which was consistent with the expected metabolite, 6-c,9-c,11-t-octadecatrienoic acid. In this instance, the relative proportions of most of the fatty acids diminished with time, except for the tri-unsaturated metabolite which increased slightly, and for oleate which increased four fold. The absolute amounts of the total fatty acids generally increased with time, up until 72 hours, and thereafter decreased. After 24 hours, 13% of the recovered CLA isomer had been converted to the desaturated metabolite. GC-mass spectrometry (GC-MS) of the methyl ester confirmed that it had the expected molecular weight.

In all further work, fatty acids were incubated with S. platensis for 96 hours to maximize the proportion of the metabolite relative to the polyunsaturated components, in order to simplify isolation and identification of structures.

10-t,12-c-octadecadienoic acid was also found to be rapidly incorporated into the lipids of S. platensis, and a metabolite (6.4% of the total fatty acids, or 18% of the conjugated acids) was formed, having a GC retention time expected for 6-c,10-t,12-c-octadecatrienoic acid. The methyl esters from the incubations of the two CLA isomers were separated first by preparative reversed-phase HPLC. In each instance, the metabolite was co-resolved with the other trienoic component, γ-linolenate. The triene fraction was then subjected to silver ion chromatography as described above (paragraph 1.7). With this procedure, a conjugated double bond system has a similar effect on retention as one isolated double bond. The metabolites emerged in a fraction expected to contain dienoic components, while the γ-linolenate was retained on the column. Each fraction was hydrolysed and converted in part to the picolinyl ester and in part to the 4,4-dimethyloxazoline (“DMOX”) derivatives which usually permit definitive determination of fatty acid structure when subjected to GC-MS (Christie, Lipids, 33: 343-353 (1998); Christie, Structural analysis of fatty acids. In Advances in Lipid Methodology—Four (Christie, W. W., ed.), pp. 119-169, Oily Press, Dundee (1997)).

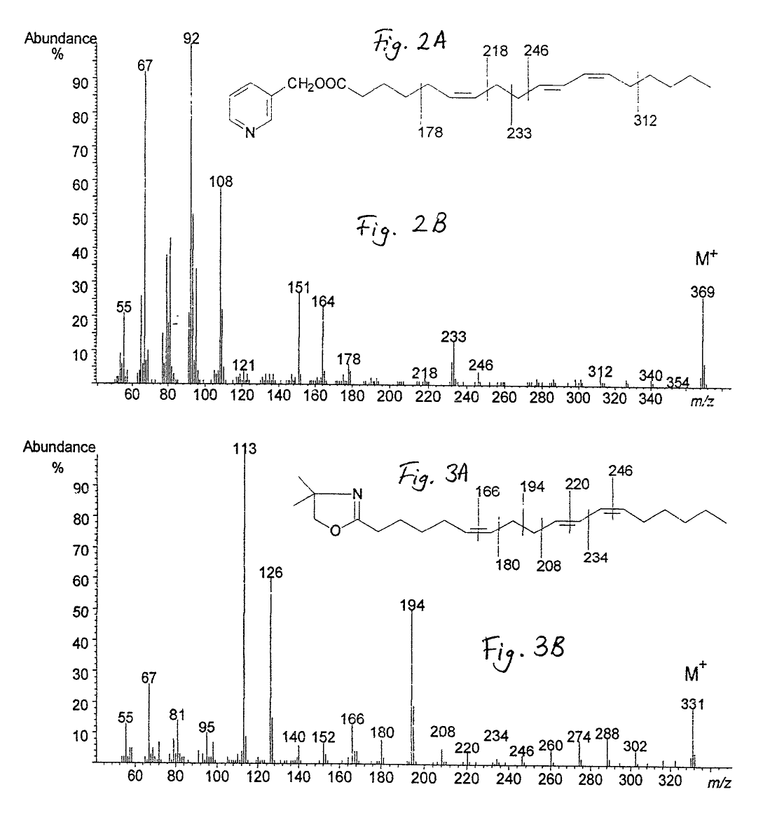

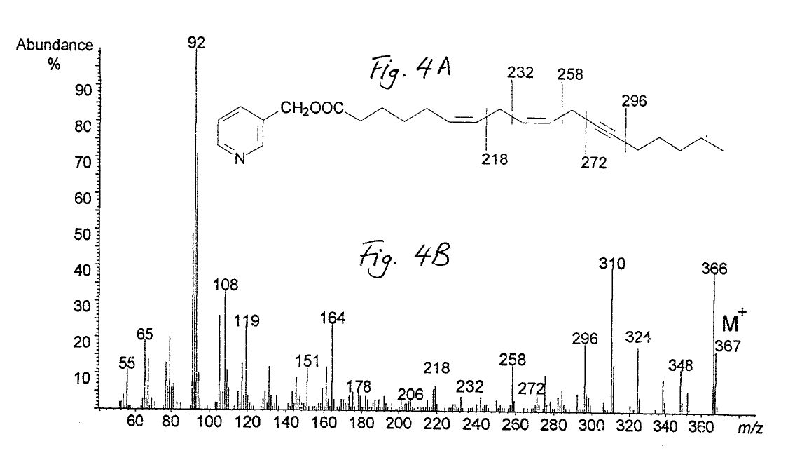

Structural formulae and mass spectra of the picolinyl ester and DMOX derivatives of 6-c,10-t,12-c-octadecatrienoic acid are illustrated in FIGS. 2A, 2B, 3A and 3B respectively. The picolinyl ester has the molecular ion at m/z=369, confirming that it has three double bonds. A distinctive ion at m/z=233 (unusual in being odd numbered) is characteristic for a -bis-methylene interrupted double bond system in positions 6 and 10. The gap of 66 atomic mass units (a.m.u.) between m/z=246 and 312 is that expected for the conjugated double bond system (plus adjacent methylene group) in positions 10,12. The double bond in position 6 is less easy to discern directly, but this region of the chromatogram is identical to that of an authentic standard of 6,10-octadecadienoate (Christie, et al, Lipids, 22: 664-666 (1987)). The mass spectrum of the dimethyloxazoline derivative corroborates the identification. The ion at m/z=194 confirms the location of the 6,10-double bond system (by extrapolation from the known 5,9-isomer (Berdeaux & Wolff, J. Am. Oil Chem. Soc., 73: 1323-1326 (1996)), and gaps of 12 a.m.u. between m/z=208 and 220, and 234 and 246 verify the location of double bonds in positions 10 and 12, respectively. The position of the double bond in position 6 is confirmed from the identity of the early part of the spectrum with that of the authentic 6-octadecenoate derivative (Spitzer, Prog. Lipid Res., 35: 387-408 (1997)). Similar spectra were obtained from the two derivatives of 6-c,9-c,11-t-octadecatrienoic acid, which were entirely consistent with the expected structure, if a little more difficult to interpret from first principles.

Mass spectrometry does not confirm the geometry of the double bonds, but they would not be expected to change during incubation.

Crepenynic acid was also taken up by S. platensis and converted to a dehydro-metabolite, octadeca-6,9-dien-12-ynoic acid, the structure and mass spectrum of the picolinyl ester of which are illustrated in FIGS. 4A and 4B respectively. The spectrum can be compared with that for picolinyl crepenynate published elsewhere. As is not uncommon for acetylenic fatty acids, an ion representing [M-1]⁺ was more abundant than the molecular ion per se. The double bond in position 9 is located by a gap of 26 a.m.u. between m/z=232 and 258, and the triple bond in position 12 by a gap of 24 a.m.u. between m/z=272 and 296. The same ions are present in the spectrum of picolinyl crepenynate, but shifted two units higher. The double bond in position 6 is less easy to discern, but the appropriate region of the spectrum is identical to that of authentic standards (and very different from other isomers). Interestingly, a C₂₀ homologue of crepenynic acid, formed by chain elongation, was also detected in the total fatty acids, though compounds of this type were not found in the control or in the incubation with CLA isomers. In this instance, dimethyloxazoline derivatives could not be prepared, but crepenynic acid can undergo rapid rearrangement under the conditions of derivatization (Christie, Chem. Phys. Lipids, 94, 35-41 (1998)). Vernonic (12-expoxy-octadeca-9-enoic) acid was not incorporated into S. platensis lipids.

The inventors have demonstrated that conjugated linoleic acid isomers especially those with the first double bond in position 9 (c) or 10 (t), added exogenously, can be desaturated in position 6 by the cyanobacterium S. platensis. No attempt has yet been made to optimize conditions to give the maximum yield of metabolites, so it is probable that the incubation conditions can be improved. 6-c,9-c, 11-t-octadecatrienoic and 6-c, 10-t, 12-c-octadecatrienoic acids may be expected to have much more pronounced biological activity than the parent conjugated linoleate isomers, as chain-elongation and further desaturation to arachidonate analogues occurs much more rapidly in animal tissues than the insertion of the first double bond in position 6. These tri-unsaturated fatty acids have never been prepared previously.

In addition, crepenynic (octadeca-9-en-12-ynoic) acid was desaturated to octadeca-6,9-dien-12-ynoic acid, which latter compound also has not been prepared previously, to the best knowledge of the inventors. This compound may have useful biological properties in itself, if elongated in animal tissues to an arachidonate analogue. The reactivity of the acetylenic bond in dehydrocrepenynate could also be utilised synthetically to prepare other novel fatty acids. It is evident that S. platensis has some potential to insert a double bond in position 6 of other polyunsaturated fatty acids that might have biological value.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, biochemistry, or related fields are intended to be within the scope of the following claims.

What is claimed is:

1. A composition comprising a conjugated octadecatrienoic acid moiety, wherein said conjugated octadecatrienoic acid moiety is desaturated at position 6.

2. The composition of claim 1, wherein said conjugated octadecatrienoic acid moiety is selected from the group consisting of c-6, c-9, t-11 octadecatrienoic acid, c-6, c-9, c-1 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid.

3. The composition of claim 1, wherein said conjugated octadecatrienoic acid moiety is a free fatty acid.

4. The composition of claim 1, wherein said conjugated octadecatrienoic acid moiety is an ester.

5. The composition of claim 4, wherein said ester is selected from the group consisting of methyl and ethyl esters.

6. The composition of claim 1, wherein said moiety is covalently attached to a glycerol.

7. The composition of claim 1, further comprising an antioxidant.

8. The composition of claim 1, further comprising a food product.

9. A pharmaceutical composition comprising an excipient and a conjugated octadecatrienoic acid moiety, wherein said conjugated octadecatrienoic acid moiety is desaturated at position 6.

10. The composition of claim 9, wherein said conjugated octadecatrienoic acid moiety is selected from the group consisting of c-6, c-9, t-11 octadecatrienoic acid, c-6, c-9, c-11 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid.

11. The composition of claim 9, wherein said conjugated octadecatrienoic acid moiety is a free fatty acid.

12. The composition of claim 9, wherein said conjugated octadecatrienoic acid moiety is an ester.

13. The composition of claim 12, wherein said ester is selected from the group consisting of methyl and ethyl esters.

14. The composition of claim 9, wherein said moiety is covalently attached to glycerol.

15. The composition of claim 9, further comprising an antioxidant.

16. A composition comprising a conjugated octadecatrienoic acid moiety made by the process comprising:
a) providing a conjugated octadecadienoic acid and a delta-6 desaturase; and
b) exposing said conjugated octadecadienoic acid to said delta-6 desaturase under conditions such that a conjugated octadecatrienoic moiety desaturated at position 6 is produced.

17. A method for preparing a conjugated octadecatrienoic acid moiety comprising:
a) providing a conjugated octadecadienoic acid and a delta-6 desaturase; and
b) exposing said conjugated octadecadienoic acid to said delta-6 desaturase under conditions such that a conjugated octadecatrienoic moiety desaturated at position 6 is produced.

18. The method of claim 17, wherein said conjugated octadecadienoic acid is selected from the group consisting of c9,t11-, t9,c11-, t9,t11-, c9,c11-, t10,c12-, c10,t12-c10,c12-, and t10,t12-octadecadienoic acids.

19. The method of claim 17, wherein said delta-6 desaturase is derived from Spirlina platensis.

20. The method of claim 17, wherein said exposing step further comprises incubating said octadecadienoic acid with a culture of Spirlina platensis.

21. The method of claim 20, wherein said Spirulina platensis is a strain selected from the group consisting of PCC8005, PCC6313, PCC7345, and PCC7939.

22. The method of claim 17, further comprising step
c) purifying said conjugated octadecatrienoic moiety.

23. A composition comprising a delta-6 desaturase, octadecadienoic acid, and an octadecatrienoic moiety.

24. The composition of claim 23, further comprising a culture of Spirlina platensis.

25. A method of altering arachidonic acid metabolism in a subject comprising:
a) providing a subject and a conjugated octadecatrienoic acid moiety, wherein said conjugated octadecatrienoic acid moiety is desaturated at position 6; and
b) administering said conjugated octadecatrienoic acid moiety to said subject under conditions such that an arachidonic acid analog is formed.

26. The method of claim 25, wherein said conjugated octadecatrienoic acid moiety is selected from the group consisting of c-6, c-9, t-11 octadecatrienoic acid, c-6, c-9, c-11 octadecatrienoic acid, c-6, t-9, t-11 octadecatrienoic acid, c-6, t-9, c-11 octadecatrienoic acid, c-6, t-10, c-12, octadecatrienoic acid, c-6, c-10, t-12, octadecatrienoic acid, c-6, t-10, t-12, octadecatrienoic acid, and c-6, c-10, c-12, octadecatrienoic acid.

27. The method of claim 25, wherein said arachidonic acid analog is selected from the group consisting of c-5, c-8, c-11, t-13 eicosatetraenoic acid and c-5, c-8, t-12, c-14 eicosatetraenoic acid.

28. The method of claim 25, wherein said conjugated octadecatrienoic moiety is provided at a dose of about 10 mg to 10 g per day.

29. The method of claim 25, wherein said conjugated octadecatrienoic moiety is provided as an acylglycerol.

30. The method of claim 25, wherein said conjugated octadecatrienoic moiety is provided as an alkyl ester.

31. The method of claim 25, wherein said conjugated octadecatrienoic moiety is provided as a salt.

32. A compound
R₁—O—R₂
wherein R₁ is selected from the group consisting of c-6, c-9, t-11 octadecatrienoyl, c-6, c-9, c-11 octadecatrienoyl, c-6, t-9, t-11 octadecatrienoyl, c-6, t-9, c-11 octadecatrienoyl, c-6, t-10, c-12, octadecatrienoyl, c-6, c-10, t-12, octadecatrienoyl, c-6, t-10, t-12, octadecatrienoyl, and c-6, c-10, c-12, octadecatrienoyl and
R₂ is selected from the group consisting of hydrogen, alkyl, glycerol, and phosphoglycerol.

33. The compound of claim 32, wherein said alkyl is selected from the group consisting of methyl, ethyl and propyl.

34. A method of producing conjugated octadecatrienoic acid comprising
a) providing gamma-linoleic acid and a catalyst; and
b) reacting said gamma-linolenic acid with said catalyst under conditions such that c6, t10, c12 octadecatrienoic acid is produced.

35. The method of claim 34, wherein said gamma-linolenic acid is provided in borage oil.
